(19) United States
(12) Patent Application Publication
(10) Pub. No.: US 2026/0354827 A1
(43) Pub. Date: Nov. 12, 2026

(54) Staple Cartridge and Buttress Loading Unit

(71) Applicant: Covidien LP, Mansfield, MA (US)

(72) Inventors: Jacob C. BARIL, North Haven, CT (US); Roanit A. FERNANDES, North Haven, CT (US); Matthew J. CHOWANIEC, North Haven, CT (US); Wojciech KISIEL, North Haven, CT (US); Anthony GADDY, North Haven, CT (US); Thomas CASASANTA, North Haven, CT (US)

(21) Appl. No.: 19/111,908

(22) PCT Filed: Sep. 26, 2023
(86) PCT No.: PCT/IB2023/059501

§ 371 Date: Mar. 14, 2025

Related U.S. Application Data
(60) Provisional application No. 63/410,323, filed on Sep. 27, 2022.

(51) Int. Cl. A61B 17/072; A61B 50/30

(52) U.S. Cl. CPC A61B 17/07292; A61B 17/07207; A61B 50/30; A61B 2017/07271

(57) ABSTRACT

A staple cartridge and buttress loading unit assembly allows a staple cartridge and a buttress material for an anvil assembly to be loaded onto a tool assembly of a stapling device in a fast and efficient manner. The staple cartridge and buttress loading unit assembly includes a staple cartridge and a loading unit. The loading unit includes a shipping cover that releasably supports the staple cartridge, a buttress sled that supports a buttress material, and a buttress shield that defines a cover cavity that is dimensioned to receive an anvil assembly of a stapling device.

### FIELD

This disclosure is directed to loading unit for loading a staple cartridge into a stapling device, and more particularly to a loading unit for loading a staple cartridge and a buttress material on a tool assembly of a stapling device.

### BACKGROUND

Various types of surgical devices used to endoscopically treat tissue are known in the art, and are commonly used, for example, for closure of tissue or organs in transection, resection, and anastomoses procedures, for occlusion of organs in thoracic and abdominal procedures, and for electrosurgically fusing or sealing tissue.

One example of such a surgical device is a surgical stapling device. Typically, surgical stapling devices include a tool assembly having an anvil assembly and a cartridge assembly. The cartridge assembly includes a channel member and a staple cartridge that is received in a cavity defined by the channel member. In certain surgical devices, the staple cartridge is releasably supported by the channel member and can be replaced within the channel member after each firing of the stapling device to facilitate reuse of the stapling device.

To reinforce a staple line in tissue, a buttress material can be provided on the staple cartridge and/or the anvil of the tool assembly. When the stapling device is fired, the buttress material is secured to the tissue to provide added strength to the tissue along the staple line. In stapling devices that include replaceable staple cartridges, replacement of the buttress material on the tool assembly can be difficult.

A continuing need exists in the art for a loading unit that can be used to load a staple cartridge quickly and easily onto a stapling device while simultaneously delivering a buttress material to the anvil assembly.

### SUMMARY

This application is directed to a staple cartridge and buttress loading unit assembly that functions to load a staple cartridge and an anvil buttress material onto a tool assembly of a stapling device in a fast and efficient manner. The staple cartridge and buttress loading unit assembly includes a staple cartridge and a loading unit. The loading unit includes a shipping cover that releasably supports the staple cartridge, a buttress sled that supports a buttress material, and a buttress shield that defines an anvil channel with the buttress sled. The anvil channel is dimensioned to receive an anvil assembly of a stapling device.

Aspects of the disclosure are directed to a loading unit including a shipping cover, a buttress sled, and a buttress shield. The shipping cover has a distal end portion, a proximal end portion, and a cartridge support member that extends from the distal end portion to the proximal end portion. The cartridge support member includes a cartridge engagement surface and resilient fingers that extend downwardly from opposite sides of the cartridge engagement surface. The resilient fingers are configured to releasably secure a staple cartridge to the shipping cover. The buttress sled has a distal end portion, a proximal end portion, and an inner surface. The distal end portion of the buttress sled includes a first mounting member that is configured to secure a first end of a buttress material to the buttress sled, and the proximal end portion of the buttress sled is pivotably secured to the shipping cover and includes a second mounting member that is configured to secure a second end of the buttress material to the buttress sled. The buttress shield is secured to the buttress sled to define an anvil channel that is configured and dimensioned to receive an anvil assembly of a surgical stapling device. The buttress shield and the buttress sled are pivotable in relation to the shipping cover between a first position in which the buttress shield and the buttress sled are in juxtaposed alignment with the shipping cover and a second position in which the buttress shield and the buttress sled are pivoted away from the shipping cover.

In aspects of the disclosure, the distal end portion of the shipping cover includes a grip portion defining a cover cavity, and the shipping cover includes locking fingers that extend into the cover cavity and are configured to engage the buttress sled to retain the buttress shield and the buttress sled in the first position.

In some aspects of the disclosure, the first mounting member is positioned within the cover cavity and includes a post.

In certain aspects of the disclosure, the second mounting member includes spaced prongs.

In aspects of the disclosure, the buttress shield defines spaced channels that extend along the cover cavity, and the buttress sled includes ribs that are received within the spaced channels to secure the buttress shield to the buttress sled.

In some aspects of the disclosure, the buttress shield defines triangular cutouts within the spaced channels, and the buttress sled includes triangular protrusions that are received within the triangular cutouts to secure the buttress sled to the buttress shield.

In certain aspects of the disclosure, the loading unit includes a biasing member that is positioned between the buttress sled and the shipping cover to urge the buttress shield and the buttress sled to the second position.

In aspects of the disclosure, a spring biasing member is supported on the second end portion of the buttress sled and includes at least one camming member.

In some aspects of the disclosure, the loading unit includes a buttress material supported on the inner surface of the buttress sled.

In certain aspects of the disclosure, the buttress material defines a first opening that receives the post of the buttress sled to secure the buttress material to the buttress sled.

In aspects of the disclosure, the post includes an edge, and the buttress defines a score that is aligned with the edge of the post.

In some aspects of the disclosure, the edge is configured to cut the buttress material through the score.

In certain aspects of the disclosure, the buttress material includes semi-circular extensions that define openings that receive the spaced prongs.

In aspects of the disclosure, the buttress material defines a second opening that is positioned within the cover cavity and is configured to receive a distal end of an anvil of a tool assembly of a stapling device.

In some aspects of the disclosure, the buttress sled and the buttress shield are integrally formed.

In certain aspects of the disclosure, the loading unit includes a slide member that is supported on the buttress shield and the buttress sled.

In aspects of the disclosure, the slide member is movable in relation to the buttress shield and the buttress sled between proximal and distal positions.

In some aspects of the disclosure, the loading unit includes a link having a first end portion pivotably coupled to the shipping cover and a second end portion pivotably coupled to the slide member.

In certain aspects of the disclosure, the slide member is movable from the proximal position to the distal position to move the buttress shield and the buttress sled from the first position to the second position.

In aspects of the disclosure, the loading unit includes a biasing member that is positioned to urge the slide member towards the first position.

In some aspects of the disclosure, the buttress shield defines an elongate slot, and the slide member includes a finger that extends through the elongate slot of the buttress shield into the anvil channel.

Other aspects of the disclosure are directed to a staple cartridge and buttress loading unit assembly that includes a staple cartridge and a loading unit. The staple cartridge includes a cartridge body, staples, and pushers. The cartridge body defines staple receiving slots, and the staples and the pushers are received within the staple receiving slots. The loading unit includes a buttress material, a shipping cover, a buttress sled, and a buttress shield. The buttress material has a distal end portion and a proximal end portion. The shipping cover has a distal end portion, a proximal end portion, and a cartridge support member that extends from the distal end portion of the shipping cover to the proximal end portion of the shipping cover. The cartridge support member includes a cartridge engagement surface and resilient fingers that extend downwardly on opposite sides of the cartridge engagement surface. The resilient fingers are engaged with the staple cartridge to releasably secure the staple cartridge to the shipping cover. The buttress sled has a distal end portion, a proximal end portion, and an inner surface. The inner surface supports the buttress material. The distal end portion of the buttress sled includes a first mounting member that is configured to secure the distal end portion of the buttress material to the buttress sled, and the proximal end portion of the buttress sled is pivotably secured to the shipping cover and includes a second mounting member that is configured to secure the proximal end portion of the buttress material to the buttress sled. The buttress shield is secured to the buttress sled. The buttress shield and the inner surface of the buttress sled define an anvil channel that is configured to receive an anvil assembly of a surgical stapling device. The buttress shield and the buttress sled are pivotable in relation to the shipping cover between a first position in which the buttress shield and the buttress sled are in juxtaposed alignment with the shipping cover and a second position in which the buttress shield and the buttress sled are pivoted away from the shipping cover.

Other aspects of the disclosure are directed to a loading unit that includes a shipping cover, a buttress sled, and a buttress shield. The shipping cover has a distal end portion, a proximal end portion, and a cartridge support member that extends from the distal end portion to the proximal end portion. The cartridge support member includes a cartridge engagement surface and resilient fingers that extend downwardly on opposite sides of the cartridge engagement surface. The resilient fingers are configured to releasably secure a staple cartridge to the shipping cover. The buttress sled has a distal end portion and a proximal end portion. The distal end portion of the buttress sled includes a first mounting member that is configured to releasably secure a first end of a buttress material to the buttress sled, and the proximal end portion of the buttress sled is pivotably secured to the shipping cover and includes a second mounting member that is configured to releasably secure a second end of the buttress material to the buttress sled. The buttress shield is secured to the buttress sled. The buttress shield and the buttress sled define an anvil channel that is configured to receive an anvil assembly of a surgical stapling device. The buttress shield and the buttress sled are pivotable in relation to the shipping cover between a first position in which the buttress shield and the buttress sled are in juxtaposed alignment with the shipping cover and a second position in which the buttress shield and the buttress sled are pivoted away from the shipping cover.

Other features of the disclosure will be appreciated from the following description.

### BRIEF DESCRIPTION OF THE DRAWINGS

Various aspects of the disclosed loading unit are described herein below with reference to the drawings, wherein:

FIG. 1 is a side perspective view from above of a staple cartridge and buttress loading unit assembly according to aspects of the disclosure in a first position;

FIG. 2 is a side perspective view from below of the staple cartridge and buttress loading unit assembly shown in FIG. 1;

FIG. 3 is a side perspective view from below of a loading unit of the staple cartridge and buttress loading unit assembly shown in FIG. 1 separated from the staple cartridge;

FIG. 4 is a side perspective view from above of the staple cartridge of the staple cartridge and buttress loading unit assembly shown in FIG. 1;

FIG. 5 is a side perspective, exploded view of the loading unit shown in FIG. 1;

FIG. 6 is a side perspective view of a buttress sled of the loading unit shown in FIG. 1 with a buttress material supported on the buttress sled;

FIG. 7 is a cross-sectional view taken along section line 7-7 of FIG. 5;

FIG. 8 is an enlarged view of the indicated area of detail shown in FIG. 5;

FIG. 9 is a bottom perspective view of the buttress sled and buttress shield of loading unit shown in FIG. 1 with parts separated;

FIG. 10 is a bottom perspective view of the buttress sled and buttress shield of the loading unit shown in FIG. 1 as the buttress shield is slid onto the buttress sled;

FIG. 11 is a bottom perspective view of the buttress sled and buttress shield of the loading unit shown in FIG. 1 with the buttress shield slid onto the buttress sled;

FIG. 12 is a cross-sectional view taken along section line 12-12 of FIG. 2;

FIG. 13 is a cross-sectional view taken along section line 13-13 of FIG. 2;

FIG. 14 is a cross-sectional view taken along section line 14-14 of FIG. 1;

FIG. 15 is a side perspective view of the staple cartridge and buttress loading unit assembly positioned adjacent a distal end of a tool assembly of a stapling device;

FIG. 15A is a perspective view from below of the anvil of the tool assembly shown in FIG. 15;

FIG. 16 is a cross-sectional view taken along section line 16-16 of FIG. 15;

FIG. 17 is a side perspective view of the tool assembly and the staple cartridge and buttress loading unit assembly shown in FIG. 15 with an anvil assembly of the tool assembly received in a channel defined between the buttress shield and the buttress sled of the loading unit;

FIG. 18 is a cross-sectional view taken along section line 18-18 of FIG. 17;

FIG. 19 is a side cross-sectional view taken through the anvil assembly and the buttress shield and the buttress sled of the loading unit shown in FIG. 16 as the anvil assembly is slid into the channel defined between the buttress shield and the buttress sled of the loading unit;

FIG. 20 is a side cross-sectional view taken through the anvil assembly and the buttress shield and the buttress sled of the loading unit with the anvil assembly received within the channel defined between the buttress shield and the buttress sled of the loading unit;

FIG. 21 is a side cross-sectional view taken through the tool assembly and the staple cartridge and buttress loading unit assembly with the anvil assembly received within the channel defined between the buttress shield and the buttress sled of the loading unit;

FIG. 22 is a cross-sectional view taken along section line 22-22 of FIG. 21;

FIG. 23 is a side view of the staple cartridge and buttress loading unit assembly supported on the anvil assembly of the tool assembly as the staple cartridge and buttress loading unit assembly is pivoted into engagement with a channel member of the tool assembly;

FIG. 24 is a cross-sectional view taken along section line of FIG. 24-24 of FIG. 23;

FIG. 25 is a cross-sectional view taken through the staple cartridge and channel of a cartridge assembly of the tool assembly shown in FIG. 23 as the staple cartridge of the staple cartridge and buttress loading unit assembly is received within the channel member of the tool assembly and disengages from the loading unit;

FIG. 26 is a side view of the loading unit shown in FIG. 16 supported on the anvil assembly of the tool assembly with the staple cartridge received in the channel member of the tool assembly and the buttress sled and shipping cover of the loading unit pivoted back towards the buttress shield of the loading unit;

FIG. 27 is a side perspective view of the loading unit of the staple cartridge and buttress loading unit assembly as the loading unit is removed from the anvil assembly of the tool assembly shown in FIG. 16;

FIG. 28 is an enlarged perspective view of a proximal portion of the anvil assembly shown in FIG. 27 with the buttress attached thereto;

FIG. 29 is a side perspective view of an alternate version of the staple cartridge and buttress loading unit assembly shown in FIG. 1 in the first position;

FIG. 30 is a side perspective, exploded view of the staple cartridge and buttress loading unit assembly shown in FIG. 29;

FIG. 31 is a cross-sectional view taken along section line 31-31 of FIG. 29;

FIG. 32 is a side perspective view of the staple cartridge and buttress loading unit assembly shown in FIG. 29 in the first position as the loading unit is slid onto an anvil assembly of a stapling device; and

FIG. 33 is a side perspective view of the staple cartridge and buttress loading unit assembly shown in FIG. 29 as the loading unit is slid onto the anvil assembly of the stapling device and the loading unit moves from the first position to in a second position to load the staple cartridge into the channel member of the stapling device.

### DETAILED DESCRIPTION

The disclosed staple cartridge and buttress loading unit assembly will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “endoscopic” is used generally to refer to endoscopic, laparoscopic, arthroscopic, and/or any other procedure conducted through a small diameter incision or cannula. Further, the term “clinician” is used generally to refer to medical personnel including doctors, nurses, surgeons, and support personnel. In addition, directional terms such as front, rear, upper, lower, top, bottom, and similar terms are used to assist in understanding the description and are not intended to limit the disclosure.

This disclosure is directed to a staple cartridge and buttress loading unit assembly for loading a staple cartridge and a buttress material for an anvil assembly onto a tool assembly of a stapling device in a fast and efficient manner. The staple cartridge and buttress loading unit assembly includes a staple cartridge and a loading unit. The loading unit includes a shipping cover that releasably supports the staple cartridge, a buttress sled that supports a buttress material, and a buttress shield that defines an anvil channel with the buttress sled that is dimensioned to receive an anvil assembly of a stapling device.

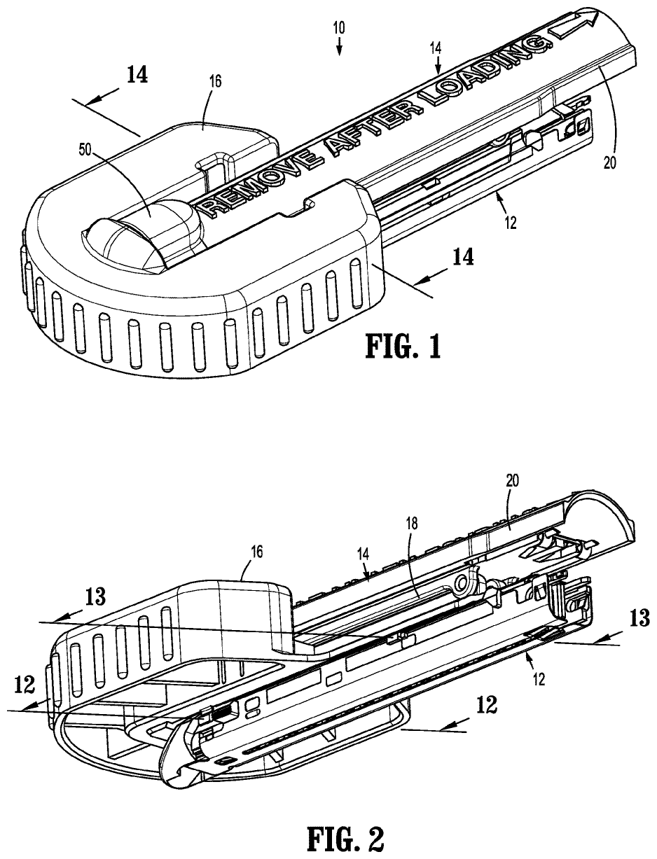

FIGS. 1-4 illustrate a staple cartridge and buttress loading unit assembly according to aspects of the disclosure shown generally as assembly 10. The assembly 10 includes a staple cartridge 12 (FIG. 4) and a loading unit 14. The loading unit 14 includes a shipping cover 16, a buttress sled 18, and a buttress shield 20. The staple cartridge is 12 releasably coupled to the loading unit 14 as described in further detail below.

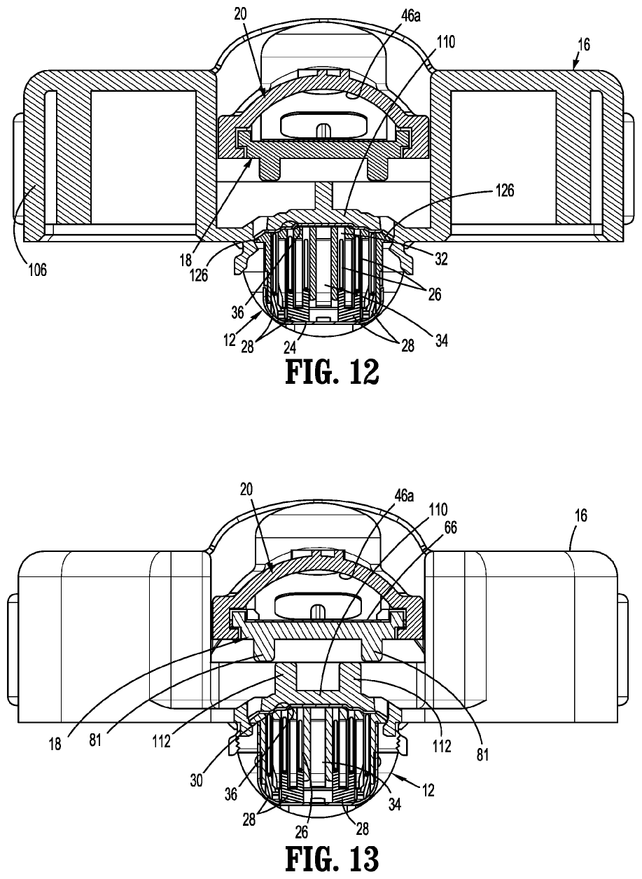

FIG. 4 illustrates the staple cartridge 12 which includes a cartridge body 22, a cartridge shield 24, staples 26 (FIG. 12), pushers 28 (FIG. 12), and a first buttress material 30. The cartridge body 22 defines staple receiving slots 32 and a knife slot 34. The staple receiving slots 32 are positioned on opposite sides of the knife slot 34 and receive the staples 26 and the pushers 28. The knife slot 34 receives a knife of a stapling device (not shown). The cartridge body 22 includes an inner surface 36 (FIG. 12) through which the knife slot 34 and the staple receiving slots 32 pass through. The inner surface 36 of the cartridge body 22 supports the first buttress material 30. In aspects of the disclosure, the first buttress material 30 is secured to the cartridge body 22 by sutures 38 although other securement devices and techniques can be used to secure the first buttress material 30 to the cartridge body 22. The cartridge body 22 includes a protrusion 40 that extends outwardly from each side of the cartridge body 22. The cartridge shield 24 is secured to a lower surface of the cartridge body 22 to retain the staples 26 and pushers 28 (FIG. 12) within the staple receiving slots 32 of the cartridge body 22.

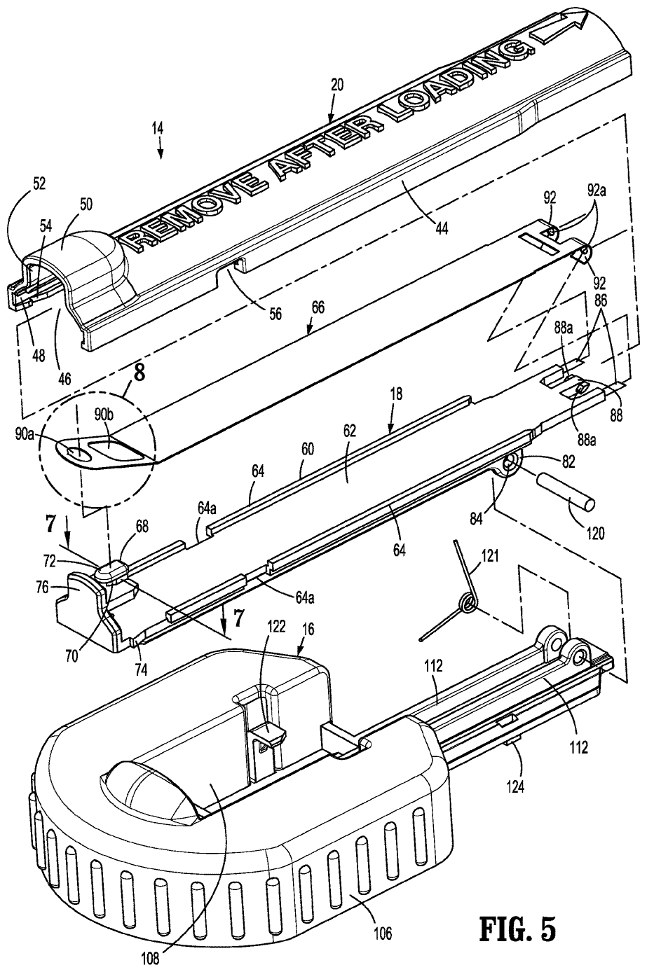

FIG. 5 illustrates an exploded view of the loading unit 14 which includes the shipping cover 16, the buttress sled 18, and the buttress shield 20. The buttress shield 20 includes an elongate body 44 that has an inner surface that defines an elongate cavity 46. The elongate body 44 of the buttress shield 20 has first and second ends and defines elongate channels 48 that extend along each side of the cavity 46 from the first end of the buttress shield 20 to the second end of the buttress shield 20. In some aspects of the disclosure, the first end of the elongate body 44 of the buttress shield 20 includes a hood 50 that is integrally formed with the elongate body 44 and defines a pocket 52. The elongate body 44 also defines an internal triangular cutout 54 that is positioned adjacent an end of each of the elongate channels 48 and a rectangular recess 56 that is positioned in opposite sidewalls of the elongate body 44 of the buttress shield 20. The rectangular recesses 56 receive the protrusions 40 of the cartridge body 22 of the staple cartridge 12 to align the staple cartridge 12 on the loading unit 14.

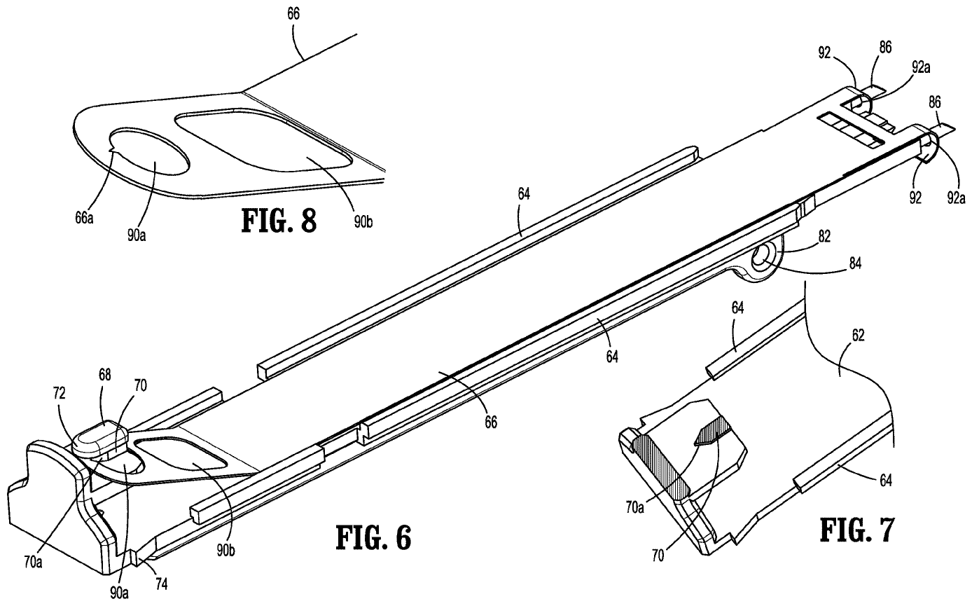

FIGS. 5-7 illustrate the buttress sled 18 which includes an elongate body 60 having a planar inner surface 62 and ribs 64 that extend along outer edges of the planar inner surface 62 along each side of the elongate body 60. The ribs 64 are received within the channels 48 of the buttress shield 20 to couple the buttress sled 18 to the buttress shield 20. The ribs 64 define recesses 64a that are aligned with the recesses 56 in the buttress shield 20 and receive the protrusions 40 (FIG. 4) of the staple cartridge 12 to align the staple cartridge 12 (FIG. 4) with the loading unit 14. The buttress sled 18 has a first end and a second end. The first end of the buttress sled 18 has a mounting member 68 for coupling a second buttress material 66 to the buttress sled 18. The mounting member 68 includes a post 70 and a cap 72 that is integrally formed with the post 70 and extends towards the first end of the buttress sled 18 past the post 70. In aspects of the disclosure, the post 70 has a triangular configuration (FIG. 8) and includes an edge 70a (FIG. 7). The first end of the buttress sled 18 includes triangular protrusions 74 that are received within the triangular cutouts 54 of the buttress shield 20 when the buttress sled 18 is slid into the channels 48 of the buttress shield 20 to secure the buttress sled 18 to the buttress shield 20. In some aspects of the disclosure, the first end of the buttress sled 18 has a cover 76 that is shaped to enclose the first end of the concavity of the buttress shield 20.

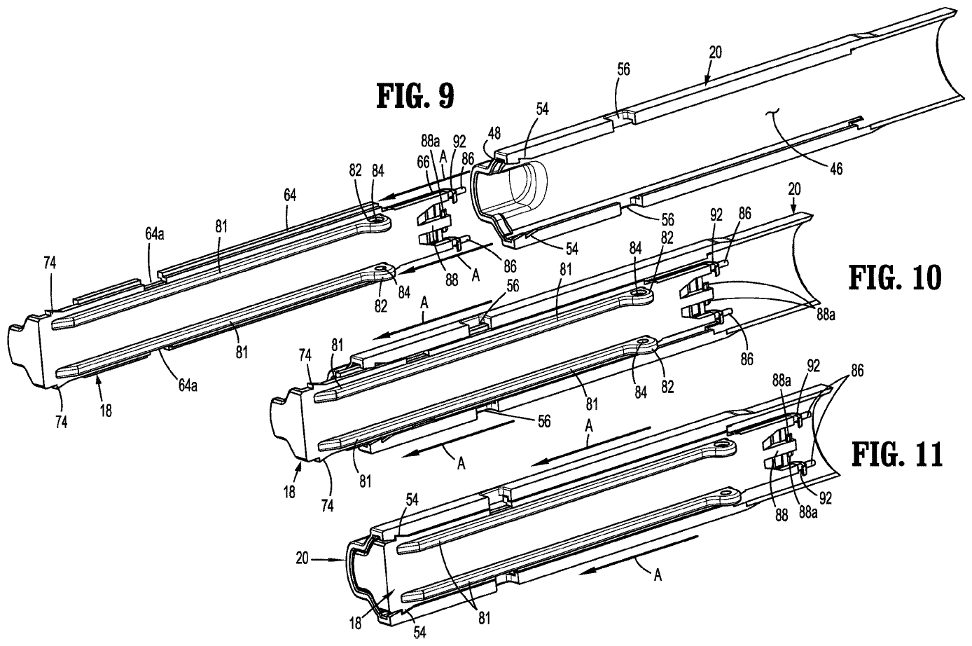

The buttress sled 18 (FIG. 9) has a lower surface that includes two downwardly longitudinal ribs 81. Each of the ribs 81 has a flange 82 positioned on the second end of the buttress sled 18 that defines a through bore 84. The flange 82 is provided to pivotally couple the buttress sled 18 to the shipping cover 16 of the loading unit 14. The second end of the buttress sled 18 also includes mounting structure configured to couple the second buttress material 66 to the second end of the buttress sled 18 and a spring biasing member 88 positioned between the spaced prongs 86. In aspects of the disclosure, the mounting structure includes spaced prongs 86. The spring biasing member 88 includes two camming members 88a that will be described in further below.

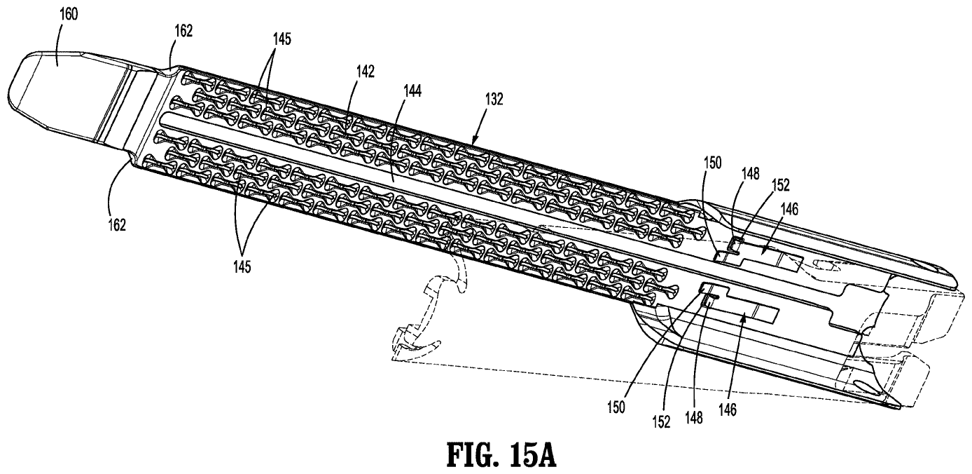

FIGS. 5-8 illustrate the second buttress material 66 which is supported on the inner planar surface 62 of the buttress sled 18 and includes a distal end portion and a proximal end portion. The distal end portion of the second buttress material 66 is angled upwardly and defines a first opening 90a and a second opening 90b that is received about the post 70 of the buttress sled 18. The second opening 90b is defined on the angled portion of the second buttress material 66 and is positioned to receive an anvil assembly 102 (FIG. 15) of a tool assembly 100 when the loading unit 14 is coupled to the tool assembly 100. As described above, the cap 72 of the mounting member 68 extends beyond the post 70 to hook the distal end portion of the second buttress material 66 to the mounting member 68 of the buttress sled 18. A portion (FIG. 8) of the second buttress material 66 defining the opening 90a includes a score or notch 66a that receives the edge 70a (FIG. 7) of the post 70 to facilitate cutting of the second buttress material 66 as described in further detail below. The proximal end portion of the second buttress material 66 includes spaced semi-circular extensions 92. Each of the semi-circular extensions 92 defines an opening 92a that receives one of the spaced prongs 86 of the buttress sled 18 to secure the proximal end portion of the second buttress material 66 to the buttress sled 18.

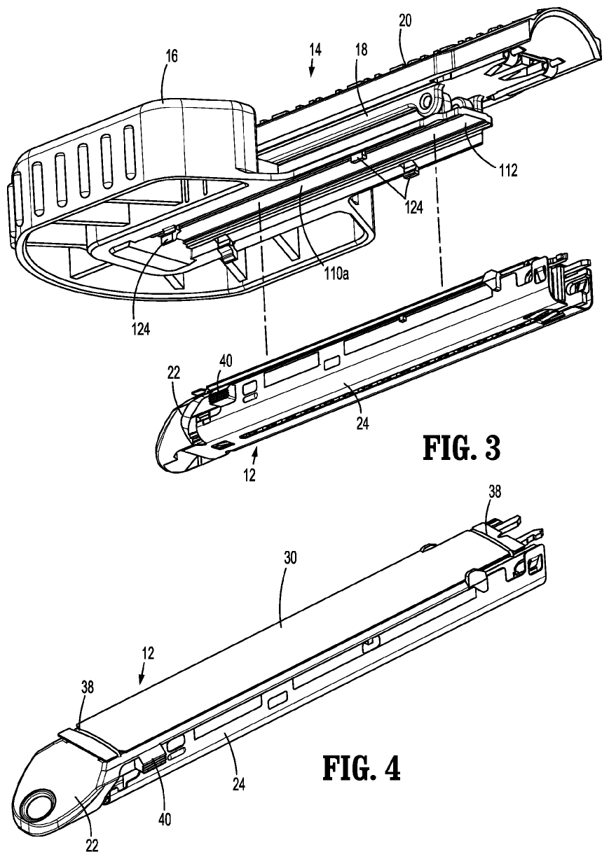

FIGS. 3, 5, and 12 illustrate the shipping cover 16 which includes a body 104 having a distal end portion that forms a grip 106 and defines a cavity 108 and a longitudinally extending cartridge support member 110 (FIG. 12). The cartridge support member 110 extends from within the cavity 108 and forms a proximal end portion of the shipping cover 16. The cartridge support member 110 includes two spaced upwardly extending ribs 112 (FIG. 5) that extend from the grip 106 along the proximal end portion of the body 104. Each of the upwardly extending ribs 112 includes a flange 114 positioned on the second end of the shipping cover 16 that defines a through bore 116. The through bores 116 receive a pivot member 120 that is also received in the openings 84 (FIG. 5) of the flanges 82 to pivotably secure the buttress sled 18 to the shipping cover 16. The buttress sled 18 and the buttress shield 20 which are secured together are pivotable in relation to the shipping cover 16 between a first position in which the buttress sled 18 and buttress shield 20 are positioned within the cavity 108 defined by the grip 106 and in longitudinal alignment with the shipping cover 16 and a second position in which the buttress sled 18 and buttress shield 20 are angled in relation to the shipping cover 16 and spaced from the cavity 108 defined by the grip 106. The pivot member 120 supports a spring or biasing member 121 that is positioned between the cartridge support member 110 of the shipping cover 16 and the buttress sled 18 to urge the buttress sled 18 to the second position. In aspects of the disclosure, the spring 121 is in the form of a torsion spring although the use of other types of biasing members is envisioned.

The body 104 of the shipping cover 16 includes two flexible locking fingers 122 that extend through the into the cavity 108 defined by the grip 106 and are aligned with the recesses 64a in the ribs 64 of the buttress sled 18. When the buttress shield 20 and the buttress sled 18 are pivoted to the first position within the cavity 108 defined by the grip 106, the locking fingers 122 flex past the buttress sled 18 and extend through the recesses 64a in the buttress sled 18 into the anvil channel 46a to a position on top of the planar inner surface 62 of the buttress sled 18 to retain the buttress sled 18 in the first position within the cavity 108 of the grip 106.

The cartridge support member 110 of the shipping cover 16 supports resilient fingers 124 (FIG. 3) that extend downwardly from the cartridge support member 110 and are positioned to engage the staple cartridge 12 to couple the staple cartridge 12 against the lower surface of the cartridge support member 110 (FIG. 12). More specifically, the resilient fingers 124 engage shoulders 126 (FIG. 12) of the cartridge body 22 of the staple cartridge 12 to releasably secure the staple cartridge 12 to the cartridge support member 110 of the shipping cover 16. When the staple cartridge 12 is supported on the cartridge support member 110 of the shipping cover 16, the cartridge support member 110 includes a cartridge engagement surface 110a that covers the outer surface of the cartridge body 22 of the staple cartridge 12 to prevent the staples 26 and the pushers 28 from falling from the staple receiving slots 32 of the cartridge body 22. In some aspects of the disclosure, the cartridge engagement surface 110a of the cartridge support member 110 defines a stepped surface that is positioned to engage the outer surface of the cartridge body 22.

FIGS. 9-11 illustrate the buttress sled 18 as the buttress sled 18 is coupled to the buttress shield 20. As briefly described above, to couple the buttress sled 18 to the buttress shield 20, the ribs 64 of the buttress sled 18 are positioned within the channels 48 of the buttress shield 20 and the buttress sled 18 is moved in relation to the buttress shield 20 in the direction indicated by arrows “A” until the triangular protrusions 74 of the buttress sled 18 are received in the triangular cutouts 54 of the buttress shield 20. When this occurs, the configuration of the protrusions 74 and the cutouts 54 locks the buttress sled 18 onto the buttress shield 20 and the buttress sled 18 encloses the cavity 46 of the buttress shield 20 to define an anvil channel 46a (FIG. 13). When the buttress sled 18 is secured to the buttress shield 20, the recesses 64a of the buttress sled 18 are aligned with the recesses 56 of the buttress shield 20.

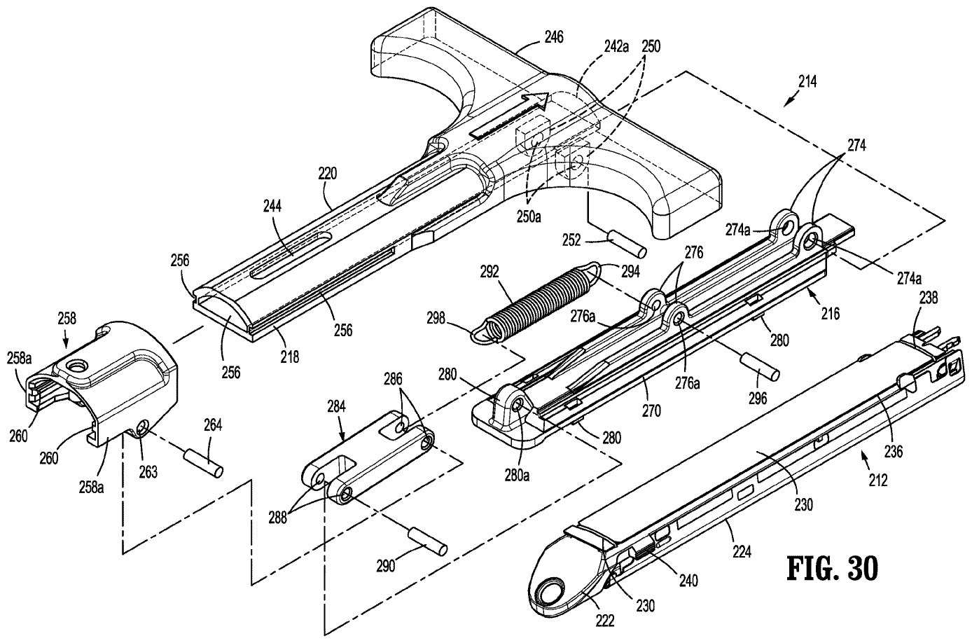

Although the buttress sled 18 and the buttress shield 20 are shown as two separate components that are coupled together, it is envisioned that the buttress sled 18 and the buttress shield 20 can be formed as an integral construction (FIG. 30) such as by molding. In aspects of the disclosure, the buttress sled 18 and the buttress shield 20 are molded from a plastic material, e.g., polyethylene, although other materials of construction are envisioned.

The assembly 10 is configured to load a staple cartridge 12 including the first buttress material 30 and the second buttress material 66 onto the tool assembly 100. The tool assembly 100 is illustrated in FIGS. 15 and 15A and includes a channel member 130 and an anvil assembly 132. The channel member 130 includes a bottom wall 134 and side walls 136 that define a cavity 138 that is dimensioned to receive the staple cartridge 12. The side walls 136 each define a recess 140 that receives one of the protrusions 40 on the cartridge body 22 of the staple cartridge 12 when the staple cartridge 12 is loaded into the cavity 138 defined by the channel member 130 of the tool assembly 100.

The anvil assembly 132 includes a staple forming surface 142 that defines a knife slot 144 and forming pockets 145 aligned in rows on each side of the knife slot 144. The anvil assembly 132 supports a pair of buttress retention springs 146 that are positioned proximally of the forming pockets 145. Each of the buttress retention springs 146 has a proximal portion secured to the anvil assembly 132 such as by welding and includes an outwardly extending hook 148 and an inwardly extending ramp member 150. The ramp members 150 are positioned transversely inwardly of the hooks 148. Each of the hooks 148 extends into a pocket 152 formed in the anvil assembly 132 when the buttress retention springs 146 are in nondeformed conditions.

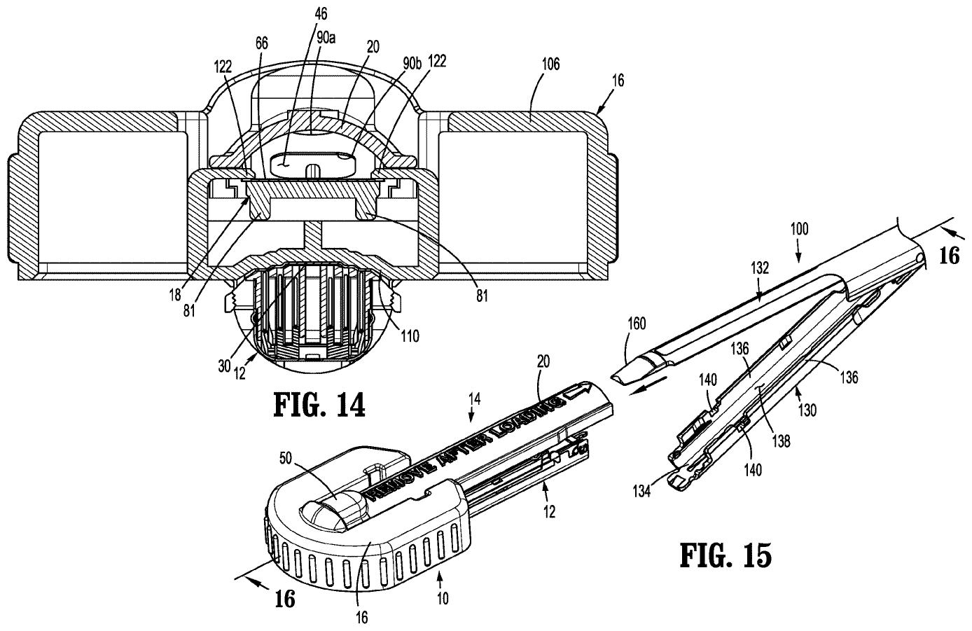

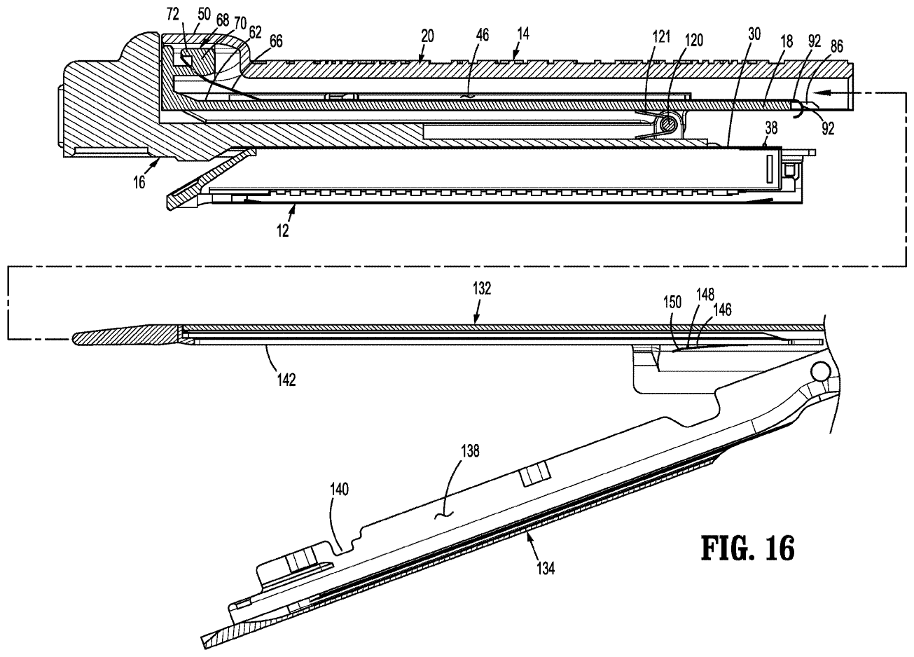

FIGS. 15 and 16 illustrate the assembly 10 and the tool assembly 100 immediately prior to loading the staple cartridge 12 and the second buttress material 66 onto the tool assembly 100. As illustrated, the buttress sled 18 is coupled to the buttress shield 20 as described above and the buttress sled is pivotally coupled to the shipping cover 16 by the pivot member 120. The buttress sled 18 with the buttress shield 20 attached is pivoted to the second position such that the buttress sled 18 and the buttress shield 20 are received in the cavity 108 of the grip 106 of the shipping cover 16. As described above, the buttress sled 18 is retained in the second position by the locking fingers 122 (FIG. 14) of the shipping cover 16. The second buttress material 66 is supported on the planar inner surface 62 of the buttress sled 18 and the post 70 of the mounting member 68 is received within the first opening 90a in the second buttress material 66.

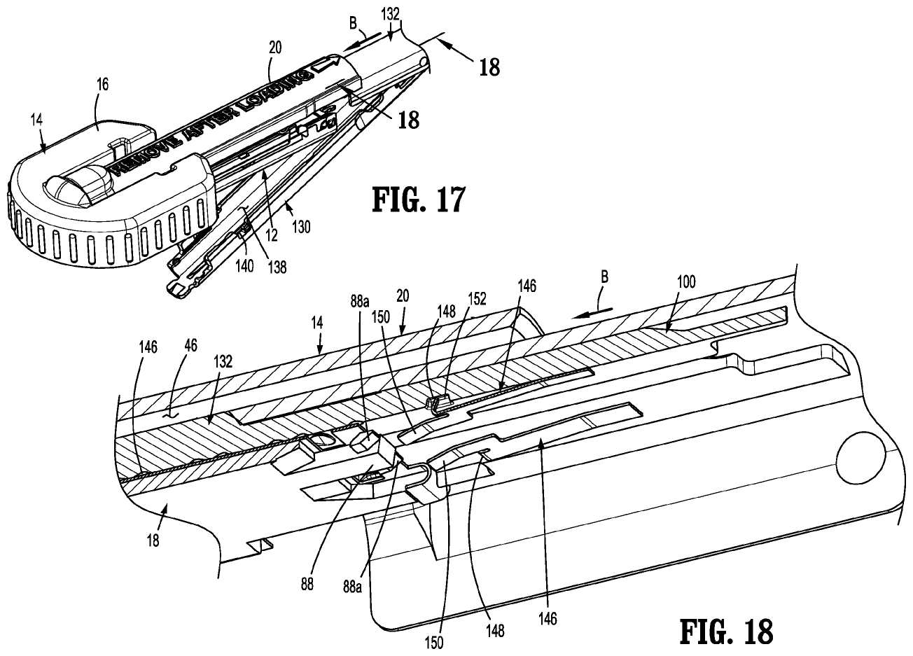

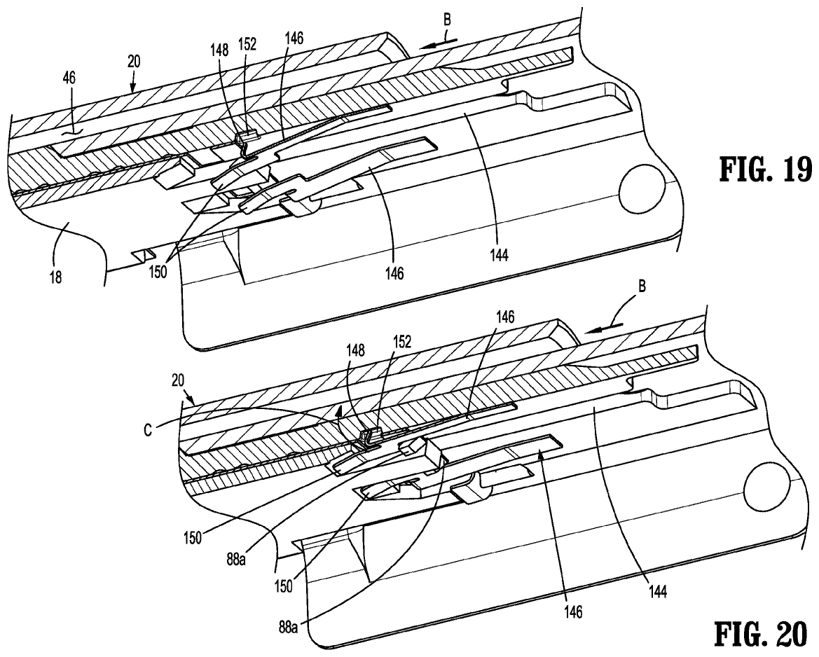

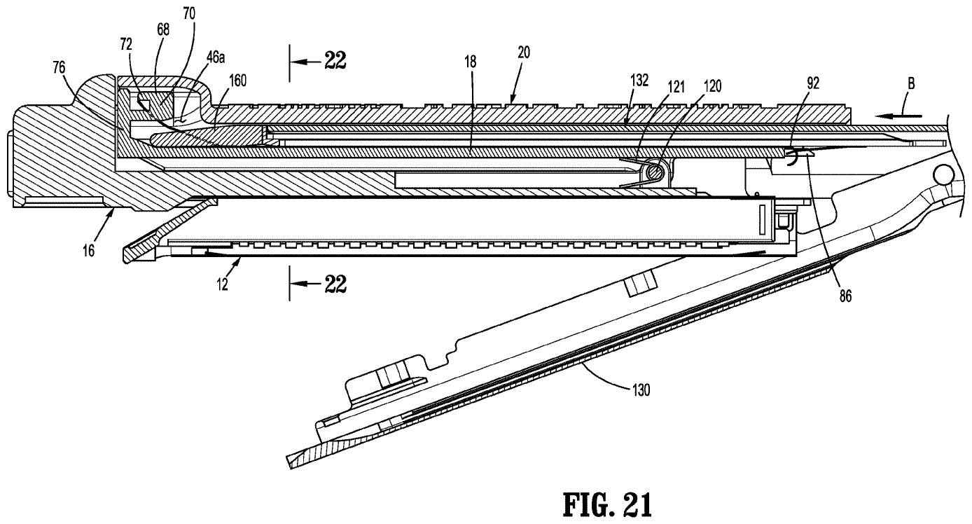

FIGS. 17-22 illustrate the anvil assembly 132 being inserted into the anvil channel 46a defined by the buttress shield 20 and the buttress sled 18. As the anvil assembly 132 is inserted into the anvil channel 46a in the direction of arrows “B” in FIGS. 18 and 19, the ramp members 150 of the buttress retention springs 146 engage the camming members 88a of the spring biasing member 88 of the buttress sled 18 and are biased inwardly to lift the hooks 148 of the buttress retention springs 146 from the pockets 152 defined in the anvil assembly 132. As the buttress sled 18 continues to move further into the anvil channel 46a defined by the buttress sled 18 and the buttress shield 20, the distal end of the anvil assembly 132 passes through the second opening 90b in the second buttress material 66. When the ramp members 150 of the buttress retention springs 146 passes over the camming member 88a of the spring biasing member 88 of the buttress sled 18, the buttress retention springs 146 return to nondeformed conditions in the direction of arrow “C” in FIG. 20 such that the hooks 148 move outwardly through the second opening 90b in the second buttress material 66 back into the pockets 152 formed in the anvil assembly 132 to secure the second buttress material 66 to the anvil assembly 132.

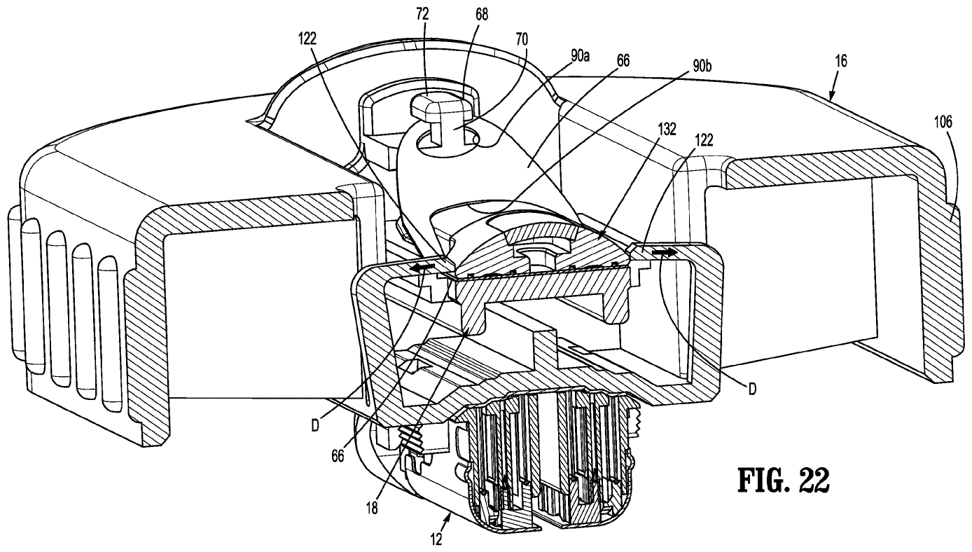

As shown in FIG. 15A, the anvil assembly 132 includes a tapered distal end portion 160 and defines cam surfaces 162 that increase in width in a proximal direction. When the anvil assembly 132 is fully received within the anvil channel 46a defined by the buttress sled 18 and the buttress shield 20, the cam surfaces 162 of the anvil assembly 132 engage the locking fingers 122 of the shipping cover 16 and bias the locking fingers 122 outwardly in the direction indicated by arrows “D” in FIG. 22 to disengage from the buttress sled 18. This facilitates movement of the buttress sled 18 and the buttress shield 20 from the first position to the second position and allows the staple cartridge 12 to pivot towards the channel member 130 of the tool assembly 100. As described above, the biasing member 121 is position to pivot the buttress sled 18 about the pivot member 120 from the first position to the second position.

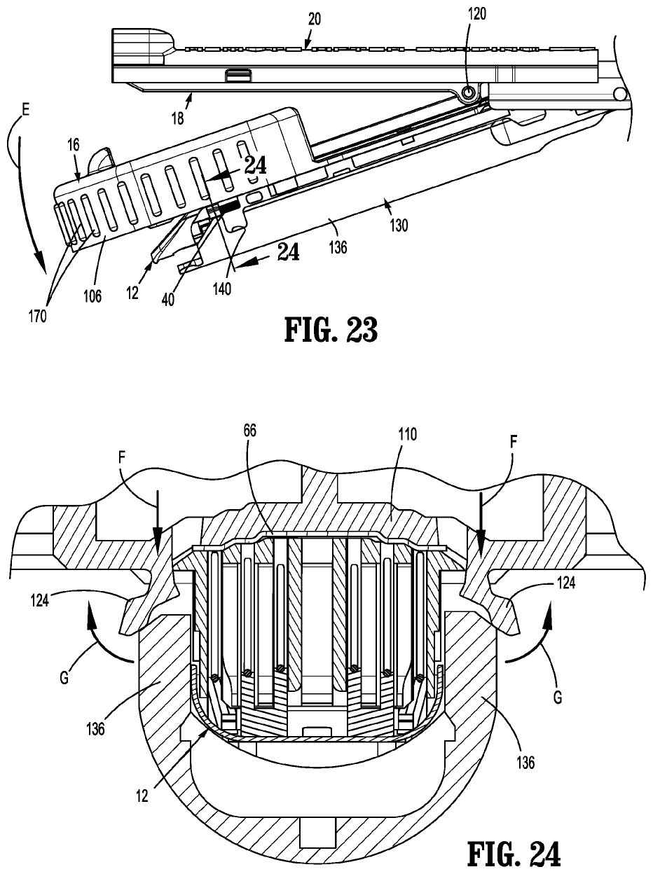

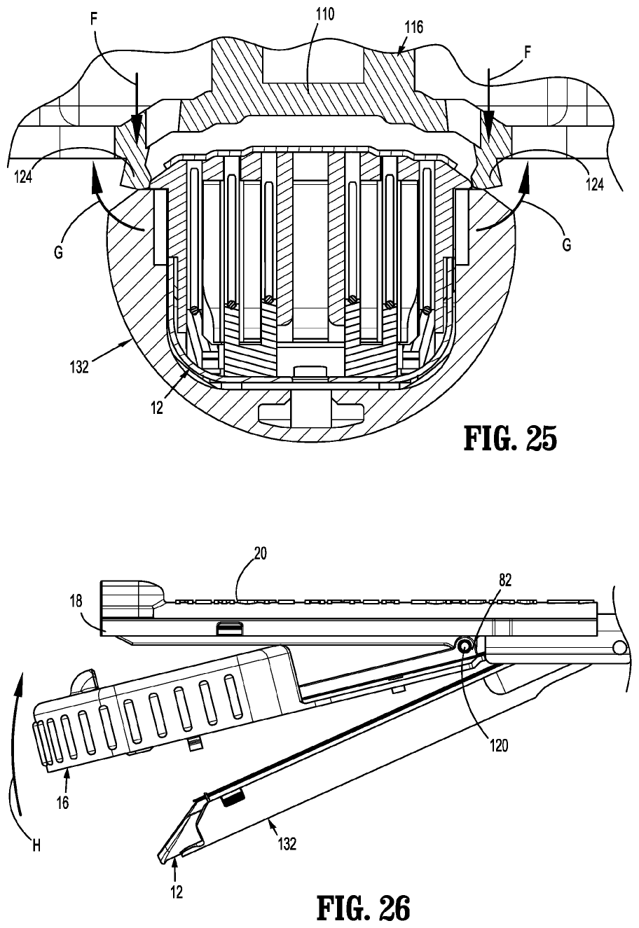

FIGS. 23-25 illustrate the staple cartridge 12 as the shipping cover 16 is pivoted about the pivot member 120 away from the buttress sled 18 and buttress shield 20 towards the channel member 130 of the tool assembly 100. Once the locking fingers 122 of the shipping cover 16 disengage from the buttress sled 18, the grip of the shipping cover 16 can be grasped and pivoted towards the channel member 130 of the tool assembly 100 in the direction indicated by arrow “E” in FIG. 23. In aspects of the disclosure, the grip 106 may include ribs 170 or other slip resistant surface texture to allow for a clinician to grasp the shipping cover more easily. As the shipping cover 16 is pivoted towards the channel member 130, the staple cartridge 12, which is supported on a lower surface of the shipping cover 16 is moved into the cavity 138 defined by the channel member 130. As the staple cartridge 12 enters the cavity 138 of the channel member 130, the protrusion 40 on the cartridge body 22 of the staple cartridge 12 is received within the recesses 140 formed in the side walls 136 of the channel member 130. As the staple cartridge 12 moves into the cavity 138 of the channel member 130 in the direction of arrows “F” in FIG. 24, the side walls 136 of the channel member 130 engage the resilient fingers 124 and urge the resilient fingers 124 outwardly in the direction of arrows “G” in FIGS. 24 and 25 to disengage the staple cartridge from the shipping cover 16.

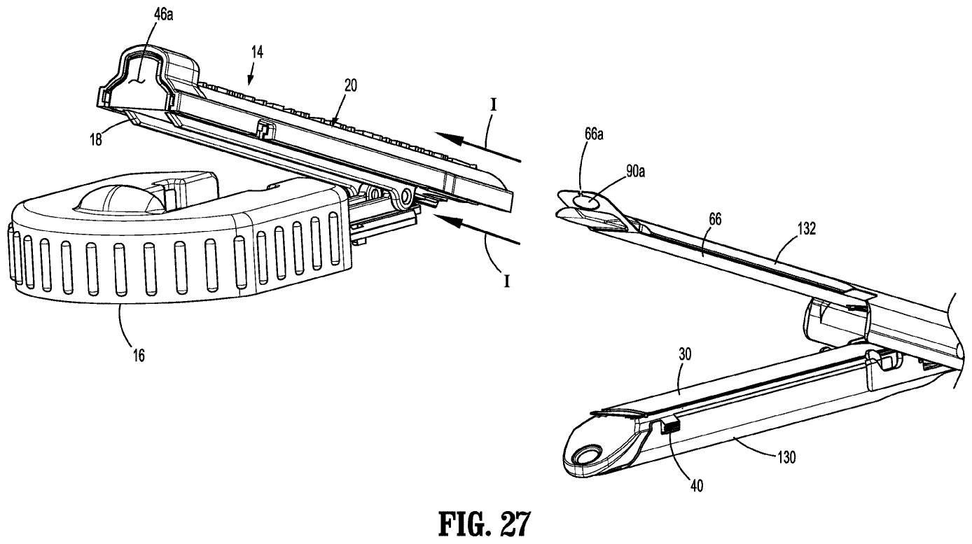

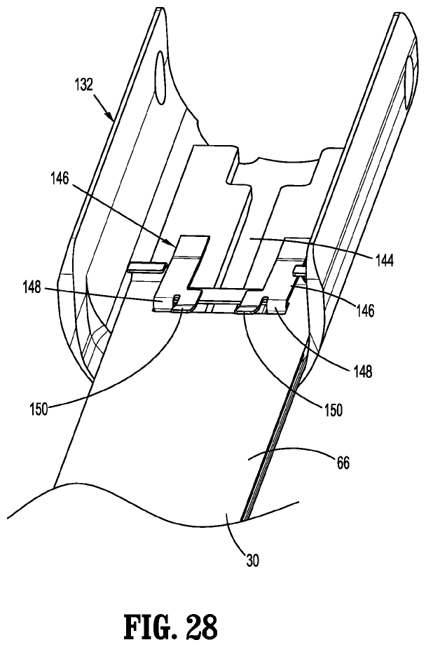

FIGS. 26-28 illustrate the tool assembly 100 and the loading unit 14 after the staple cartridge 12 is loaded into the channel member 130 of the tool assembly 100. Once the staple cartridge 12 is loaded into the channel member 130 of the tool assembly 100, the shipping cover 16 of the loading unit 14 can be pivoted in the direction of arrow “H” in FIG. 26 back towards the buttress sled 18 and the buttress sled 18 and buttress shield 20 can be slid as a unit from the anvil assembly 132 of the tool assembly 100 in the direction of arrows “I” in FIG. 27. As the buttress sled 18 and buttress shield 20 are slid from the anvil assembly 132, the edge 70a of the post 70 of the buttress sled 18 cuts through the score 66a in the end of the second buttress material 66 to allow the buttress material 66 to separate from the buttress sled 18. When the buttress sled 18 is removed from the anvil assembly 132, the spaced prongs 86 of the buttress sled 18 are pulled through the openings 92a in the semi-circular extensions 92 (FIG. 5) of the second buttress material 66. When the buttress sled 18 is removed from the anvil assembly 132, the buttress retention springs 146 of the anvil assembly 132 secure one end of the second buttress material 66 to one end of the anvil assembly 132 (FIG. 28) and receipt of the distal end of the anvil assembly 132 within the second opening 90b of the second buttress material 66 secures the other end of the anvil assembly 132.

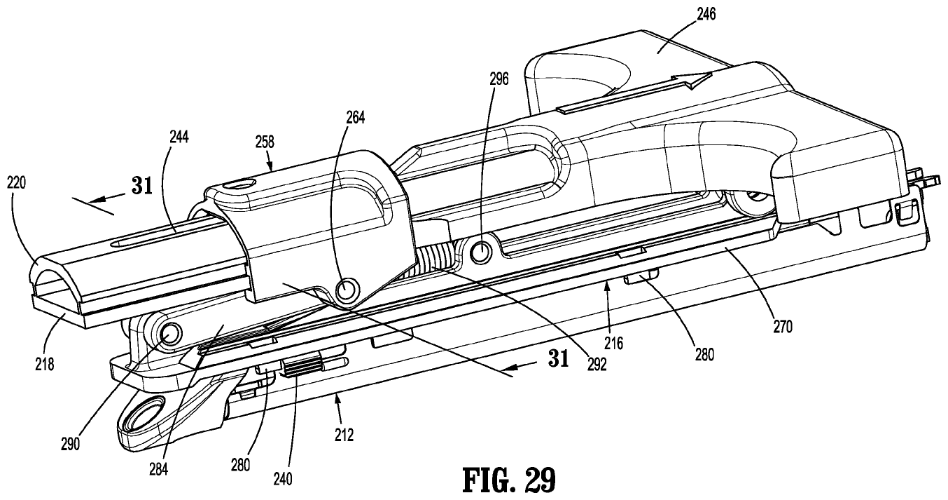

FIGS. 29-31 illustrate an alternate version of the staple cartridge and buttress loading unit assembly shown generally as assembly 200. The assembly 200 includes a staple cartridge 212 and a loading unit 214. The loading unit 214 includes a shipping cover 216, a buttress sled 218, and a buttress shield 220. The buttress sled 218 and the buttress shield 220 are integrally formed, and the staple cartridge is 12 releasably coupled to the loading unit 14 as described in further detail below.

The staple cartridge 212 is like the staple cartridge 12 (FIG. 4) and includes a cartridge body 222, a cartridge shield 224, staples and pushers (not shown), and a first buttress material 230. As described above regarding staple cartridge 12, the cartridge body 222 defines staple receiving slots (not shown) that receive the staples and the pushers. The cartridge body 222 includes an inner surface 236 through which a knife slot (not shown) and the staple receiving slots extend. The inner surface 236 of the cartridge body 222 supports the first buttress material 230. In aspects of the disclosure, the first buttress material 230 is secured to the cartridge body 222 by sutures 238 although other securement devices and techniques can be used to secure the first buttress material 230 to the cartridge body 22. The cartridge body 222 includes a protrusion 240 that extends outwardly from each side of the cartridge body 222. The cartridge shield 224 is secured to a lower surface of the cartridge body 222 to retain the staples and pushers within the staple receiving slots of the cartridge body 222.

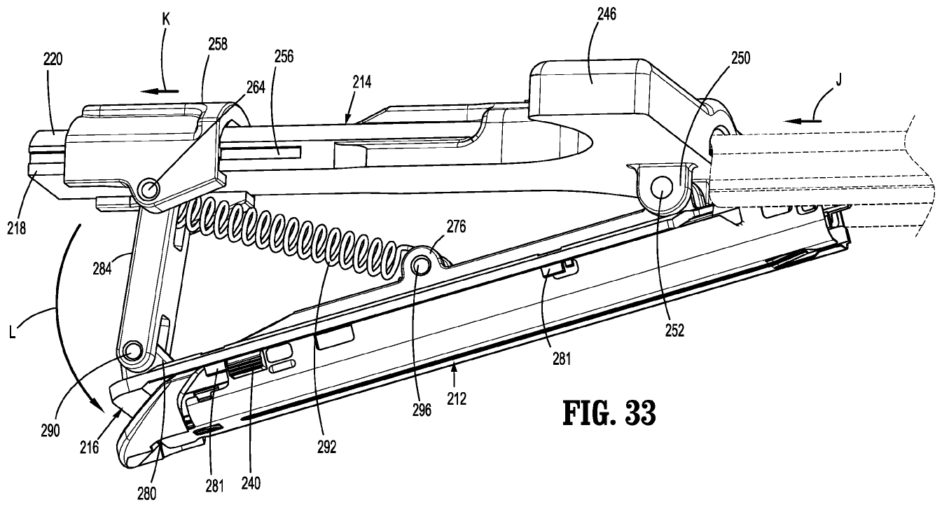

FIG. 30 illustrates an exploded view of the loading unit 214 which includes the shipping cover 216, the buttress sled 218, and the buttress shield 220. As described above, the buttress sled 218 and the buttress shield 220 are integrally formed and define an anvil channel 242 that has an open proximal end 242a. The buttress shield 220 defines an elongate slot 244 that extends through the buttress shield 220 and communicates with the anvil channel 242. The buttress shield 220 and/or the buttress sled 218 have proximal ends that define a grip 246 that can be grasped by a clinician to facilitate placement of the loading unit 214 onto a tool assembly 100 (FIG. 33) of a surgical stapling device.

The buttress sled 218 includes a lower surface (FIG. 30) that includes two downwardly extending flanges 250 that are positioned on the proximal portion of the buttress sled 218. Each of the flanges 250 defines a through bore 250a that receive a pivot member 252. The flanges 250 and the pivot member 252 are provided to pivotally couple the buttress sled 218 to the shipping cover 216 of the loading unit 214 as described in further detail below. The buttress sled 218 and/or the buttress shield 220 defines channels 256 that extend along a distal portion of side walls of the buttress sled 218 and/or the buttress shield 220.

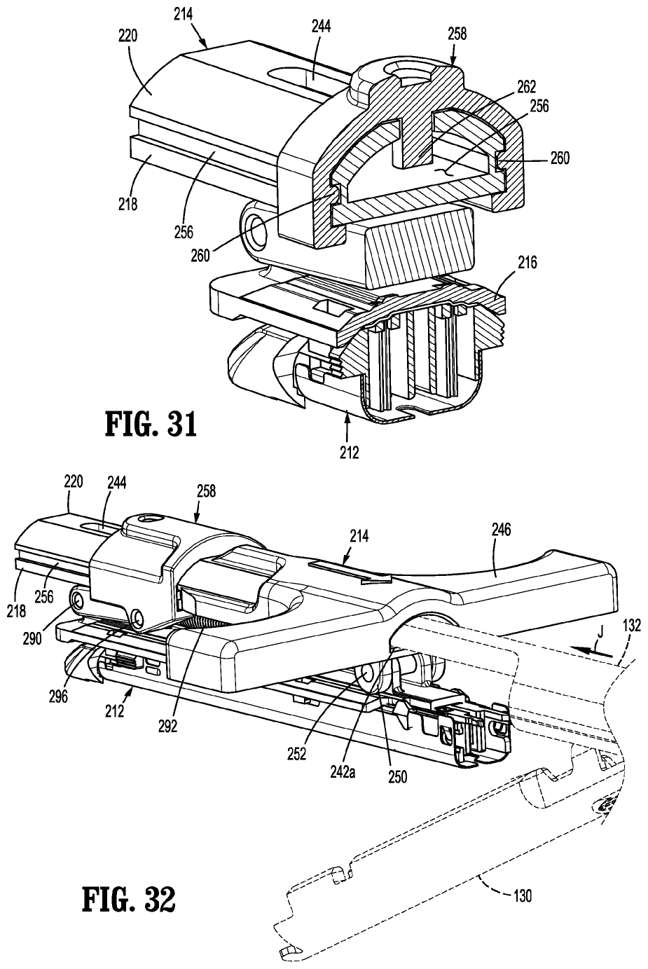

The loading unit 214 includes a slide member 258 that has a substantially C-shaped configuration and is slidable about the outer surface of the buttress shield 220. The slide member 258 includes side walls 258a having inwardly extending protrusions 260 that are received in the channels 256 of the buttress sled 218 and/or the buttress shield 220. The slide member 258 also includes a downwardly extending finger 262 (FIG. 31) that is received within the elongate slot 244 of the buttress shield 220 and extends into the anvil channel 256. The side walls 258a also defines two through bores 263 that receive a pivot member 264 as described below.

The shipping cover 216 includes a longitudinally extending cartridge support member 270 that includes an inner surface having two spaced ribs 272. Each of the ribs 272 includes a proximal flange 274 and a central flange 276. The proximal and central flanges 274 and 276 define through bores 274a, 276a. The cartridge support member 270 also includes a distal flange 280 that defines a through bore 280a. The pivot member 252 extends through the through bores 274a of the shipping cover 216 and through the through bores 250a (FIG. 30) of the buttress sled 218 to pivotally couple the proximal portion of the buttress sled 218 to the proximal portion of the shipping cover 216. The cartridge support member 270 of the shipping cover 216 includes resilient fingers 281 (FIG. 29) that extend downwardly from the cartridge support member 270 and are positioned to engage the staple cartridge 212 to couple the staple cartridge 212 against the outer surface of the cartridge support member 270 (FIG. 29). More specifically, the resilient fingers 281 engage shoulders, e.g., shoulders 126 (FIG. 12) of the cartridge body 222 of the staple cartridge 212 to releasably secure the staple cartridge 212 to the cartridge support member 270 of the shipping cover 216. When the staple cartridge 212 is supported on the cartridge support member 270 of the shipping cover 216, the cartridge support member 270 includes a cartridge engagement surface 270a that covers the outer surface of the cartridge body 222 of the staple cartridge 212 to prevent the staples and the pushers from falling from the staple receiving slots of the cartridge body 222. In some aspects of the disclosure, the cartridge engagement surface 270a of the cartridge support member 270 defines a stepped surface (FIG. 31) that is positioned to engage the outer surface of the cartridge body 222.

The loading unit 214 includes a link 284 that couples the shipping cover 216 to the buttress shield 218. In aspects of the disclosure, the link 284 is H-shaped and defines proximal openings 286 and distal openings 288. The proximal openings 286 receive the pivot member 264 which also extends through the through bores 263 of the slide member 258 to pivotably couple the link 284 to the slide member 258. The distal openings 288 receive a pivot member 290 which also extends through the through bore 280a in the distal flange 280 of the shipping cover 216 to pivotably couple the link 284 to the distal portion of the shipping cover 216.

The loading unit 214 includes a biasing member 292 that is positioned to urge the slide member 258 proximally on the buttress shield 220 and to urge the shipping cover 216 towards a first position in which the shipping cover 216 is aligned with the buttress sled 218. In aspects of the disclosure, the biasing member 292 includes a coil spring that has a first loop 294 that is received about a pivot member 296 that is received through the through bores 276a of the central flanges 276 of the shipping cover 216, and a second loop 298 that is received about the pivot member 264 of the slide member 258. Alternately, other types biasing members can be used to urge the shipping cover towards the first position.

Although the internal configuration of the buttress sled 218 and the buttress shield 220 for supporting and delivering a buttress material to an anvil assembly are not described in detail herein, it is envisioned that the features described above regarding the buttress sled 18 and the buttress shield 20 can be included in the buttress sled 218 and the buttress shield 220 to support and deliver the second material 66 (FIG. 5) to the anvil assembly 132.

FIGS. 32 and 33 illustrate the assembly 200 as the loading unit 214 is used to load the second buttress material 66 (FIG. 5) onto the anvil assembly 132 and load the staple cartridge 212 into the channel member 130 of the tool assembly 100 (FIG. 15) of a stapling device. Prior to use, the loading unit 200 with the staple cartridge 212 attached to the shipping cover 216 is urged to the first position in which the shipping cover 216 is longitudinally aligned with the buttress sled 218 and the buttress shield 220. In use, the assembly 200 is positioned to align the distal end of the anvil assembly 132 with the open proximal end 242a of the anvil channel 256 defined between the buttress sled 218 and the buttress shield 220. The anvil assembly 132 is inserted into the anvil channel 256. This can be accomplished by pulling the grip 246 proximally to move the anvil assembly 132 within the anvil channel 256 in the direction of arrow “J” in FIG. 32. As the anvil assembly 132 moves through the anvil channel 256, the distal end of the anvil assembly 132 engages the finger 262 (FIG. 31) of the slide member 258 to move the slide member 258 distally along the buttress sled 218 and the buttress cover 220 in the direction of arrow “K” in FIG. 33 against the urging of biasing member 292. As the slide member 258 moves in the direction of arrow “K”, the link 284 pivots to pivot the shipping cover 216 downwardly from the first position to the second position in the direction of arrow “L” in FIG. 33 to position the staple cartridge 212 within the channel member 130 of the tool assembly 100. As the staple cartridge 212 enters the channel member 130, the resilient fingers 281 of the shipping cover 216 engage side walls of the channel member 130 to release the staple cartridge 212 from the shipping cover.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

The invention may be described by reference to the following numbered paragraphs:-

1. A loading unit comprising:
a shipping cover having a distal end portion, a proximal end portion, and a cartridge support member extending from the distal end portion to the proximal end portion, the cartridge support member including a cartridge engagement surface and resilient fingers extending downwardly on opposite sides of the cartridge engagement surface, the resilient fingers configured to releasably secure a staple cartridge to the shipping cover;
a buttress sled having a distal end portion, a proximal end portion, and an inner surface, the distal end portion of the buttress sled including a first mounting member configured to secure a first end of a buttress material to the buttress sled, and the proximal end portion of the buttress sled pivotably secured to the shipping cover and including a second mounting member configured to secure a second end of the buttress material to the buttress sled; and
a buttress shield secured to the buttress sled, the buttress shield and the inner surface of the buttress sled defining an anvil channel configured to receive an anvil assembly of a surgical stapling device, the buttress shield and the buttress sled pivotable in relation to the shipping cover between a first position in which the buttress shield and the buttress sled are in juxtaposed alignment with the shipping cover and a second position in which the buttress shield and the buttress sled are pivoted away from the shipping cover.

2. The loading unit of claim 1, wherein the distal end portion of the shipping cover includes a grip defining a cover cavity, the shipping cover including locking fingers that extend into the cover cavity and are configured to engage the buttress sled to retain the buttress shield and the buttress sled in the first position.

3. The loading unit of claim 2, wherein the first mounting member is positioned within the cover cavity and includes a post.

4. The loading unit of claim 1, wherein the second mounting member includes spaced prongs.

5. The loading unit of claim 2, wherein the buttress shield defines spaced channels that extend along the cover cavity, and the buttress sled includes ribs that are received within the spaced channels to secure the buttress shield to the buttress sled.

6. The loading unit of claim 5, wherein the buttress shield defines triangular cutouts within the spaced channels, and the buttress sled includes triangular protrusions that are received within the triangular cutouts to secure the buttress sled to the buttress shield.

7. The loading unit of claim 1, further including a biasing member positioned between the buttress sled and the shipping cover, the biasing member positioned to urge the buttress shield and the buttress sled to the second position.

8. The loading unit of claim 1, further including a spring biasing member supported on the second end portion of the buttress sled, the spring biasing member including at least one camming member.

9. The loading unit of claim 1, further including a buttress material supported on the inner surface of the buttress sled.

10. The loading unit of claim 3, wherein the buttress material defines a first opening that receives the post of the buttress sled to secure the buttress material to the buttress sled.

11. The loading unit of claim 10, wherein the post includes an edge, and the buttress defines a score that is aligned with the edge of the post, the edge configured to cut the buttress material through the score.

12. The loading unit of claim 4, wherein the buttress material includes semi-circular extensions, each of the semi-circular extensions defining an opening that receives one of the spaced prongs.

13. The loading unit of claim 10, wherein the buttress material defines a second opening that is positioned within the cover cavity and is configured to receive a distal end of an anvil of a tool assembly of a stapling device.

14. The loading unit of claim 1, wherein the buttress sled and the buttress shield are integrally formed.

15. The loading unit of claim 1, further including a slide member supported on the buttress shield and the buttress sled, the slide member movable in relation to the buttress shield and the buttress sled between proximal and distal positions.

16. The loading unit of claim 15, further including a link having a first end portion pivotably coupled to the shipping cover and a second end portion pivotably coupled to the slide member, wherein the slide member is movable from the proximal position to the distal position to move the buttress shield and the buttress sled from the first position to the second position.

17. The loading unit of claim 16, further including a biasing member positioned to urge the slide member to the first position.

18. The loading unit of claim 17, wherein the buttress shield defines an elongate slot, and the slide member includes a finger that extends through the elongate slot into the anvil channel.

19. A staple cartridge and buttress loading unit assembly comprising:
a staple cartridge including a cartridge body, staples, and pushers, the cartridge body defining staple receiving slots, the staples and the pushers received within the staple receiving slots;
a loading unit, the loading unit including
   a buttress material having a distal end portion and a proximal end portion;
   a shipping cover having a distal end portion, a proximal end portion, and a cartridge support member extending from the distal end portion of the shipping cover to the proximal end portion of the shipping cover, the cartridge support member including a cartridge engagement surface and resilient fingers extending downwardly on opposite sides of the cartridge engagement surface, the resilient fingers engaged with the staple cartridge to releasably secure the staple cartridge to the shipping cover;
   a buttress sled having a distal end portion, a proximal end portion, and an inner surface, the inner surface supporting the buttress material, the distal end portion of the buttress sled including a first mounting member configured to secure the distal end portion of the buttress material to the buttress sled, and the proximal end portion of the buttress sled pivotably secured to the shipping cover and including a second mounting member configured to secure the proximal end portion of the buttress material to the buttress sled; and
   a buttress shield secured to the buttress sled, the buttress shield and the inner surface of the buttress sled defining an anvil channel configured to receive an anvil assembly of a surgical stapling device, the buttress shield and the buttress sled pivotable in relation to the shipping cover between a first position in which the buttress shield and the buttress sled are in juxtaposed alignment with the shipping cover and a second position in which the buttress shield and the buttress sled are pivoted away from the shipping cover.

20. The staple cartridge and buttress loading unit assembly of claim 19, wherein the distal end portion of the shipping cover includes a grip defining a cover cavity, and the shipping cover includes locking fingers that extend into the cover cavity and are configured to engage the buttress sled to retain the buttress shield and the buttress sled in the first position.

21. The staple cartridge and buttress loading unit assembly of claim 20, wherein the first mounting member is positioned within the cover cavity and includes a post, and the second mounting member includes spaced prongs, the distal end portion of the buttress material defining a first opening that receives the post of the buttress sled to secure the buttress material to the buttress sled, and the proximal end portion of the buttress material includes semi-circular extensions, each of the semi-circular extensions defining an opening that receives one of the spaced prongs.

22. The staple cartridge and buttress loading unit assembly of claim 20, wherein the buttress shield defines spaced channels that extend along the cover cavity, and the buttress sled includes ribs that are received within the spaced channels to secure the buttress shield to the buttress sled.

23. The staple cartridge and buttress loading unit assembly of claim 20, further including a biasing member positioned between the buttress sled and the shipping cover, the biasing member positioned to urge the buttress shield and the buttress sled to the second position.

24. The staple cartridge and buttress loading unit assembly of claim 19, further including a spring biasing member supported on the second end of the buttress sled, the spring biasing member including at least one camming member.

25. The staple cartridge and buttress loading unit assembly of claim 19, wherein the buttress sled and the buttress shield are integrally formed.

26. The staple cartridge and buttress loading unit assembly of claim 19, further including a slide member supported on the buttress shield and the buttress sled, the slide member movable in relation to the buttress shield and the buttress sled between proximal and distal positions.

27. The staple cartridge and buttress loading unit assembly of claim 26, further including a link having a first end portion pivotably coupled to the shipping cover and a second end portion coupled to the slide member, wherein the slide member is movable from the proximal position to the distal position to move the buttress shield and the buttress sled from the first position to the second position.

28. The staple cartridge and buttress loading unit assembly of claim 27, further including a biasing member positioned to urge the slide member to the first position.

29. The staple cartridge and buttress loading unit assembly of claim 28, wherein the buttress shield defines an elongate slot, and the slide member includes a finger that extends through the elongate slot into the anvil channel.

30. A loading unit comprising:
a shipping cover having a distal end portion, a proximal end portion, and a cartridge support member extending from the distal end portion to the proximal end portion, the cartridge support member including a cartridge engagement surface and resilient fingers extending downwardly on opposite sides of the cartridge engagement surface, the resilient fingers configured to releasably secure a staple cartridge to the shipping cover;
a buttress sled having a distal end portion and a proximal end portion, the distal end portion of the buttress sled including a first mounting member configured to releasably secure a first end of a buttress material to the buttress sled, and the proximal end portion of the buttress sled pivotably secured to the shipping cover and including a second mounting member configured to releasably secure a second end of the buttress material to the buttress sled; and
a buttress shield secured to the buttress sled, the buttress shield and the buttress sled defining an anvil channel configured to receive an anvil assembly of a surgical stapling device, the buttress shield and the buttress sled pivotable in relation to the shipping cover between a first position in which the buttress shield and the buttress sled are in juxtaposed alignment with the shipping cover and a second position in which the buttress shield and the buttress sled are pivoted away from the shipping cover.

1. A buttress loading unit comprising:
a shipping cover having a distal end portion, a proximal end portion, and a cartridge support member extending from the distal end portion to the proximal end portion, the cartridge support member including a cartridge engagement surface and resilient fingers extending downwardly on opposite sides of the cartridge engagement surface, the resilient fingers configured to releasably secure a staple cartridge to the shipping cover;
a buttress sled having a distal end portion, a proximal end portion, and an inner surface, the distal end portion of the buttress sled including a first mounting member configured to secure a first end of a buttress material to the buttress sled, and the proximal end portion of the buttress sled pivotably secured to the shipping cover and including a second mounting member configured to secure a second end of the buttress material to the buttress sled; and
a buttress shield secured to the buttress sled, the buttress shield and the inner surface of the buttress sled defining an anvil channel configured to receive an anvil assembly of a surgical stapling device, the buttress shield and the buttress sled pivotable in relation to the shipping cover between a first position in which the buttress shield and the buttress sled are in juxtaposed alignment with the shipping cover and a second position in which the buttress shield and the buttress sled are pivoted away from the shipping cover.

2. The buttress loading unit of claim 1, wherein the distal end portion of the shipping cover includes a grip defining a cover cavity, the shipping cover including locking fingers that extend into the cover cavity and are configured to engage the buttress sled to retain the buttress shield and the buttress sled in the first position.

3. The buttress loading unit of claim 2, wherein the first mounting member is positioned within the cover cavity and includes a post.

4. The buttress loading unit of claim 3, wherein the buttress material defines a first opening that receives the post of the buttress sled to secure the buttress material to the buttress sled.

5. The buttress loading unit of claim 4, wherein the post includes an edge, and the buttress defines a score that is aligned with the edge of the post, the edge configured to cut the buttress material through the score.

6. The buttress loading unit of claim 2, wherein the buttress shield defines spaced channels that extend along the cover cavity, and the buttress sled includes ribs that are received within the spaced channels to secure the buttress shield to the buttress sled.

7. The buttress loading unit of claim 6, wherein the buttress shield defines triangular cutouts within the spaced channels, and the buttress sled includes triangular protrusions that are received within the triangular cutouts to secure the buttress sled to the buttress shield.

8. The buttress loading unit of claim 1, further comprising a buttress material, wherein the second mounting member includes spaced prongs, and the buttress material includes semi-circular extensions, each of the semi-circular extensions defining an opening that receives one of the spaced prongs.

9. The buttress loading unit of claim 8, wherein the buttress material includes semi-circular extensions, each of the semi-circular extensions defining an opening that receives one of the spaced prongs.

10. The buttress loading unit of claim 1, further comprising a biasing member positioned between the buttress sled and the shipping cover, the biasing member positioned to urge the buttress shield and the buttress sled to the second position.

11. The buttress loading unit of claim 1, further comprising a spring biasing member supported on the buttress sled, the spring biasing member including at least one camming member.

12. The buttress loading unit of claim 1, further comprising a buttress material supported on the inner surface of the buttress sled.

13. A staple cartridge and buttress loading unit assembly comprising:
a staple cartridge including a cartridge body, staples, and pushers, the cartridge body defining staple receiving slots, the staples and the pushers received within the staple receiving slots;
a loading unit, the loading unit including
a buttress material having a distal end portion and a proximal end portion;
a shipping cover having a distal end portion, a proximal end portion, and a cartridge support member extending from the distal end portion of the shipping cover to the proximal end portion of the shipping cover, the cartridge support member including a cartridge engagement surface and resilient fingers extending downwardly on opposite sides of the cartridge engagement surface, the resilient fingers engaged with the staple cartridge to releasably secure the staple cartridge to the shipping cover;
a buttress sled having a distal end portion, a proximal end portion, and an inner surface, the inner surface supporting the buttress material, the distal end portion of the buttress sled including a first mounting member configured to secure the distal end portion of the buttress material to the buttress sled, and the proximal end portion of the buttress sled pivotably secured to the shipping cover and including a second mounting member configured to secure the proximal end portion of the buttress material to the buttress sled; and
a buttress shield secured to the buttress sled, the buttress shield and the inner surface of the buttress sled defining an anvil channel configured to receive an anvil assembly of a surgical stapling device, the buttress shield and the buttress sled pivotable in relation to the shipping cover between a first position in which the buttress shield and the buttress sled are in juxtaposed alignment with the shipping cover and a second position in which the buttress shield and the buttress sled are pivoted away from the shipping cover.

14. The staple cartridge and buttress loading unit assembly of claim 13, wherein the distal end portion of the shipping cover includes a grip defining a cover cavity, and the shipping cover includes locking fingers that extend into the cover cavity and are configured to engage the buttress sled to retain the buttress shield and the buttress sled in the first position.

15. The staple cartridge and buttress loading unit assembly of claim 14, wherein the first mounting member is positioned within the cover cavity and includes a post, and the second mounting member includes spaced prongs, the distal end portion of the buttress material defining a first opening that receives the post of the buttress sled to secure the buttress material to the buttress sled, and the proximal end portion of the buttress material includes semi-circular extensions, each of the semi-circular extensions defining an opening that receives one of the spaced prongs.

16. The staple cartridge and buttress loading unit assembly of claim 14, wherein the buttress shield defines spaced channels that extend along the cover cavity, and the buttress sled includes ribs that are received within the spaced channels to secure the buttress shield to the buttress sled.

17. The staple cartridge and buttress loading unit assembly of claim 14, further comprising a biasing member positioned between the buttress sled and the shipping cover, the biasing member positioned to urge the buttress shield and the buttress sled to the second position.

18. The staple cartridge and buttress loading unit assembly of claim 13, further comprising a spring biasing member supported on the buttress sled, the spring biasing member including at least one camming member.

19. The staple cartridge and buttress loading unit assembly of claim 13, wherein the buttress sled and the buttress shield are integrally formed.

20. A buttress loading unit comprising:
a shipping cover having a distal end portion, a proximal end portion, and a cartridge support member extending from the distal end portion to the proximal end portion, the cartridge support member including a cartridge engagement surface and resilient fingers extending downwardly on opposite sides of the cartridge engagement surface, the resilient fingers configured to releasably secure a staple cartridge to the shipping cover;
a buttress sled having a distal end portion and a proximal end portion, the distal end portion of the buttress sled including a first mounting member configured to releasably secure a first end of a buttress material to the buttress sled, and the proximal end portion of the buttress sled pivotably secured to the shipping cover and including a second mounting member configured to releasably secure a second end of the buttress material to the buttress sled; and
a buttress shield secured to the buttress sled, the buttress shield and the buttress sled defining an anvil channel configured to receive an anvil assembly of a surgical stapling device, the buttress shield and the buttress sled pivotable in relation to the shipping cover between a first position in which the buttress shield and the buttress sled are in juxtaposed alignment with the shipping cover and a second position in which the buttress shield and the buttress sled are pivoted away from the shipping cover.
